(19) United States
(12) Patent Application Publication
(10) Pub. No.: US 2025/0125024 A1
(43) Pub. Date: Apr. 17, 2025

(54) Method and software for medical multimodel translation

(71) Applicant: Andy Liu, Calgary (CA)

(72) Inventor: Andy Liu, Calgary (CA)

(21) Appl. No.: 18/445,553

(22) Filed: Oct. 13, 2023

(51) Int. Cl. G16H 10/60

(52) U.S. Cl. CPC G16H 10/60

(57) ABSTRACT

The present disclosure provides a method, application, and system for medical multimodal translation. A multimodal translation processor has at least three modules that process medical inputs in one language and translates them to multiple modes of medical outputs in another language: medical terminology, layman explanations, and related visual media data. The translation processor connects and retrieves information from medical data. Each module has a trained AI or non-AI model that automatically processes input and generates the respective outputs in bi-directions. A patient may also take a photo or video of their symptoms or select the symptom area for processing and determining the correct medical term, explanation, and related visual media. The multimodal translation processor can be made into a software application in a device or a system. Patients can use the application to book their appointment, describe symptoms, retrieve medical history, and communicate with a medical professional/doctor during their visit.

### CROSS-REFERENCE TO RELATED APPLICATIONS

This non-provisional application claims the benefit of U.S. Provisional Patent Application No. 63/414,171, filed Oct. 7, 2022. The entire disclosure of the above application is incorporated herein by reference.

### FIELD

The present disclosure is in the field of language translation, medical communication, medical comprehension, visual media, software applications, artificial intelligence, machine learning, and especially, a method and software application for translating medical information into multimodal media and text.

### BACKGROUND

Many countries have diverse groups of people that speak many different languages and dialects. Some conversations may involve people that do not speak a common language or are not fluent enough, which makes communication difficult. To get around this issue, translation (via a person, device, or software application) can be used to facilitate communication between people that speak different languages or dialects. This is useful in many situations, but it is particularly important in medical situations, where patient-doctor communication is key for providing proper medical care. A doctor is one type of medical professional that has a high degree of medical knowledge and skill. Other types of medical professionals are nurses, medical technicians, assistants, volunteers, etc. For the purpose of brevity, the term ‘doctor’ may be hereinafter used interchangeably with the term ‘medical professional’. Doctors need to understand what a patient is describing in order to know his problems and symptoms; likewise, Patients need to correctly understand what doctor diagnoses and treatments.

A human translator can translate on behalf of both parties. This can be done both in-person or via a virtual appointment. More importantly, the human translator can help interpret any word's meaning, cultural context, or technical knowledge within the conversation. However, a human translator may not always be available to be an option. Moreover, there may not be a language translator having knowledge of specific languages, or the translator may know only the language but not the necessary medical knowledge. For example, a hospital may have a staff member that can translate between Spanish and English, but a doctor-patient conversation may require a translation between Chinese and English. If no one is available to translate this specific conversation, medical care cannot be properly provided.

On the other hand, a machine translator may provide language translation at any time. The patient and doctor can speak or type what they want to say, which then gets translated by the machine using text or speech synthesis (e.g., text-to-speech or vice versa). However, the accuracy of the translation can vary and may not be consistent. It also does not take background, personality, interpretation ability, and cultural context into consideration. Moreover, both parties are dependent on a device, reliable software, or an Internet connection (e.g., cellular data or WiFi signal).

An additional problem with existing medical translation is when medical terminologies are used, the terms are only familiar to the doctor. A patient would find them unfamiliar and incomprehensible. Some prior arts translate medical terms into word-for-word sentences. There is no explanation given alongside the translated terms. So, a doctor must explain the diagnosis to the patient in layman's words. If the translation is not available or accurate, the doctor's explanation cannot even be reliably communicated to the patient, let alone in a way the patient can understand.

Moreover, the patient may not have the knowledge or skills required to properly explain their problem or symptom effectively or sufficiently to the doctor. Without the ability to properly express everything in the doctor's language, many details may be left out, and then may lead to a wrong diagnosis and treatment. The patient may rely on the doctor to guide them step-by-step to show up close or, in the case of a virtual appointment, a picture of the symptom, but details may still be left out without the proper translation. The image quality, lighting, resolution, and other various parameters may greatly affect the quality of the diagnosis on top of potential translation issues. Therefore, patients need a tool to reliably and consistently show their symptoms. Besides, patients and medical professionals may also need a visual example to better describe and understand a symptom or a diagnosis. In such a case, visual media can be used to further show examples of a symptom or diagnosis via an image or a YouTube video on the Internet. However, there is a limited amount of material that can be shown for specific symptoms, particularly if such media is from an existing database or a set number of images on a doctor's hard drive. A Google search can be done to find more visual examples, but it is time-consuming and has to be manually searched by the user (either patient or doctor). Because of the limitations mentioned above, there is an increased need for an automatic method, system, and/or software for easy and better medical translation.

The present disclosure provides a method and system for better-translating information. The method, software application, and system improve the following: (1) improved mutual communication between users, especially medical doctors and patients who speak different languages or dialects; (2) improved comprehension of medical knowledge at different levels of skills and knowledge (e.g., description of symptoms and problems; comprehension of diagnosis and cure); (3) increases translation automation, efficiency, and accuracy; (4) medical data is more accessible, secure, and easy to maintain.

### SUMMARY

The present disclosure provides a method, software application, and system for medical multimodal translation. The purpose is to improve communication between two users-a first and second user that speaks different languages. Particularly if the first user is a medical professional (e.g., a doctor) and the second is a patient. The present disclosure comprises a new multimodal translation processor. The multimodal translation processor takes various medical inputs in one language and converts them to various modes of medical outputs in another language. The various modes include but are not limited to text (terminology in text and/or explanation in text), audio, pictures, videos, hyperlinks, PDF documents, VR, 3D models, and other media/information formats and representations. The present disclosure has the following novel aspects: (1) a method for automatic multimodal translation of communication from one language to another language with appropriate terminology, explanations, visual media, and more; (2) a method for automatic translation of multimedia and selection inputs to the appropriate terminology, explanations, and visual media in the desired language; (3) a method and system of the creation and training of all the necessary AI models for a translation processor implementing the said multimodal translation methods, especially in medical care industry; (4) a software application of said translation processor within a device and its overall application system to provide better interaction and more efficient communication between users especially medical professionals and patients; (5) medical data security feature implemented in the system.

The first aspect of the present disclosure involves a method for automatic translation of medical terminology from one language (first language) to another (second language) among multimodal formats via a multimodal translation processor. The multimodal translation processor is typically implemented within an application software of a device that takes medical inputs in the form of text or audio (e.g., voice recording) in the first language. The multimodal formats include but are not limited to text, audio (e.g., voice), pictures, videos, hyperlinks, and PDF documents, etc. Selection input (e.g., selection of a picture area by the user, choice of language, and formats for medical inputs) is also taken into account within the translation processor. The multimodal translation processor has three functional modules: a medical terminology module or basic module for determining the correct medical terminology of a symptom, diagnosis, or cure/treatment in a different language; an explanation module or expanded module for generating an explanation of the terminology in greater detail or an easier to understand way; a visual media module or advanced module for searching up and generating related pictures, videos, or other media formats. The visual content may help users to understand the topic even better. The multimodal translation processor uses a medical database and artificial intelligence (AI) methods and tools to help generate the required medical outputs. Medical output comes out in the form of a second language (either text or audio), along with visual output. This multimodal translation is bi-directional. The text, audio, explanation, and media outputs in the second language can also be translated back to multimodal formats in the first language at the input side.

The second aspect involves a method for translating multimedia (images, video, etc.) and selection inputs (from selecting part of one or more multimedia inputs) to the appropriate medical terminologies and explanations in the desired language. Unlike in the first aspect, the visual and selection inputs can also be directly processed by the translation processor. A core part of the second aspect involves a visual media smart search based on the provided multimedia and selection inputs to find similar pictures/media of the symptom or diagnosis. In one exemplary embodiment, a patient may take a picture of their symptom with their device for the system to translate; the patient has the option to take a picture or video showing their symptom. The patient can select the area where the symptom, for example, a lesion, is present or let the software automatically detect it for them. The translation processor uses advanced image processing to segment and recognizes the lesion through AI visual search. After the segmentation, it makes an outline of the symptom that is processed through the visual module to carry out the visual media search and get the required medical outputs, particularly visual output. The module also generates the corresponding explanation and terminologies texts describing the visual outputs.

The third aspect involves the implementation of the creation and training of all the necessary AI models. Each module of the multimodal translation processor may have an AI model that can automatically generate the appropriate medical output. The AI models can be trained and tested using a set of medical input data. The AI remembers any accurate known output from the training and applies it for clinical use. That way, the translation processor can make an accurate translation prediction and give related media that correlate with a symptom and diagnosis. Moreover, at least one AI model can be used with the second aspect in automatically capturing a symptom area.

The fourth aspect involves one embodiment of the implementation that a software application uses said medical translation processor methods. It includes the software app within a device and its overall system to provide better interaction and more efficient communication between medical professionals and patients. A patient and a medical professional can use the software application on their respective device to interact with one another. The software application connects to medical data, which is connected to a network and application server. The application has a graphical user interface with several pages that are typical of a medical appointment application, such as login, appointment setting, medical history, language setting, etc. The patient can fill in their medical history before the appointment. One page of the application allows the patient to point out the affected body area, pick out a symptom, and describe its intensity, frequency, and start date. The doctor uses medical data to determine a diagnosis, treatment, and medication.

The fifth aspect involves a security system architecture and feature implementation for viewing and transferring data over an application server securely. Medical information can be transferred and stored in the app and server; the data is encrypted and needs a private key. The patient uses a password to receive their medical data and the provided private key to decrypt it. A private key is a variable in cryptography that is used with an algorithm to encrypt and decrypt data. Such keys are usually a string of numbers or letters that are stored in a file and processed through a cryptographic algorithm. The medical data is then downloaded to the server and database. The user logs out to re-encrypt the data.

The medical translation system and method overcome the prior art limitations and provide an accurate, simple, automatic, and user-friendly means for translating doctor-patient communication from one language to another in a multimodal fashion. The present disclosure improves the following: (1) improved communication between users; (2) improved patient comprehension of a symptom, diagnosis, and other relevant medical information; (3) increased convenience thanks to AI and software applications; (4) data is more secure thanks to the security system features.

### BRIEF DESCRIPTION OF THE DRAWINGS

The accompanying drawings, which are included to provide a further understanding of the invention and are incorporated in and constitute a part of this application, illustrate embodiment(s) of the present disclosure and, together with the description, serve to explain the principle of the invention. For simplicity and clarity, the figures of the present disclosure illustrate a general manner of construction of various embodiments. Descriptions and details of well-known features and techniques may be omitted to avoid unnecessarily obscuring the discussion of the present disclosure's described embodiments. It should be understood that the elements of the figures are not necessarily drawn to scale. Some elements' dimensions may be exaggerated relative to other elements for enhancing the understanding of described embodiments. In the drawings:

FIG. 1 illustrates a general method for multimodal translation between medical inputs and outputs.

FIG. 2 illustrates how multiple modes of the inputs in a first language are translated to multiple modes of medical outputs via the multimodal translation processor.

FIG. 3 illustrates an exemplary block diagram of AI processing within a multimodal translation processor.

FIG. 4 illustrates an exemplary system structure design for doctor-patient communication using the multimodal translation software in the present disclosure.

FIG. 5 illustrates an exemplary design for the media and selection inputs to be translated into output by the multimodal translation software.

FIG. 6 illustrates the graphical user interface of the app using the translation system of the present disclosure.

FIG. 7 illustrates the symptom description pages used for the translation system of the present disclosure.

FIG. 8 illustrates a process flowchart of the system's general use.

FIG. 9 illustrates a flowchart for securely maintaining medical data.

FIG. 10 illustrates a flowchart for doctor-patient communication using the application in the present disclosure.

FIG. 11 illustrates a block diagram of an exemplary hardware and software implementation within a device.

FIG. 12 illustrates a flowchart for user input for showing a physical symptom using the system of the present disclosure.

FIG. 13 illustrates a flowchart of the expanded/advanced translation process using the selection input in the present disclosure.

FIG. 14 illustrates a flowchart for the training and translation of the AI models within the translation processor of the present disclosure.

### DETAILED DESCRIPTION

The present disclosure provides a method, software application, and system for medical multimodal translation. Various examples of the present invention are shown in the figures. However, the present invention is not limited to the illustrated embodiments. In the following description, specific details are mentioned to give a complete understanding of the present disclosure. However, it may likely be evident to a person of ordinary skill in the art; hence, the present disclosure may be applied without mentioning these specific details. The present disclosure is represented as a few embodiments; however, the disclosure is not necessarily limited to the particular embodiments illustrated by the figures or description below.

The language employed herein only describes particular embodiments; however, it is not limited to the disclosure's specific embodiments. The terms “they”, “he/she”, or “he or she” are used interchangeably because “they”, “them”, or “their” are considered singular gender-neutral pronouns. The terms “comprise” and/or “comprising” in this specification are intended to specify the presence of stated features, steps, operations, elements, and/or components; however, they do not exclude the presence or addition of other features, steps, operations, elements, components, or groups.

Unless otherwise defined, all terminology used herein, including technical and scientific terms, have the same definition as what is commonly understood by a person of ordinary skill in the art, typically to whom this disclosure belongs. It will be further understood that terms, such as those defined in commonly used dictionaries, should be interpreted as having the same meaning as defined in the context of the relevant art and the present disclosure. Such terms should not be construed in an overly strict sense unless explicitly described herein. It should be understood that multiple techniques and steps are disclosed in the description, each with its own benefit. Each technique or step can also be utilized in conjunction with a single, multiple, or all of the other disclosed techniques or steps. For brevity, the description will avoid repeating each possible combination of the steps unnecessarily. Nonetheless, it should be understood that such combinations are within the scope of the disclosure. Reference will now be made in detail to some embodiments of the present invention, examples of which are illustrated in the accompanying figures.

The present disclosure has a few notable aspects of novelty. In the first aspect of the present disclosure, there is a method for translating from text or audio (e.g., voice) of one language (a first language) to different presentation formats of another language (a second language). The method involves medical inputs being processed through a multimodal translation processor to be translated as multimodal medical outputs. A first user, either a patient or doctor, talks or types down what they want to say. The inputs can be a variety of multimodal formats that include but are not limited to text (terminology in text and/or explanation in text), audio, pictures, videos, hyperlinks, PDF documents, VR, 3D models, and other media/information formats and representations. The multimodal translation processor processes this input; any non-text input has individual preprocessors that convert the input data to text. For example, input in the form of voiced dialogue is preprocessed into text using a speech recognition preprocessor or first preprocessor. Any visual input in the form of images or video can be preprocessed into text for language translation, mainly through a visual media preprocessor or a second preprocessor. Selection input can be made manually by the user or automatically by the software to exemplify what should be processed. In one exemplary scenario, the patient and/or doctor provides the languages and formats of their choice for language translation. In another exemplary scenario, the selection input may be a particular area within an image or any highlighted text within a software application. In the case of language translation for this aspect, the selection input gets preprocessed into text through a selection preprocessor or third preprocessor.

The multimodal translation processor comprises three modules that take the (preprocessed) inputs and generate basic, expanded, and advanced descriptions that are translated to become medical outputs. The basic, expanded, and advanced descriptions in the present disclosure are representations of the medical output formats. The translation processor modules include: a medical terminology module that takes whatever is being described (e.g., symptom) and determines the appropriate medical terminology relevant to the dialogue, such as a symptom, diagnosis, cure, or treatment. Hereinafter, the term ‘medical terminology module’ is interchangeable with ‘basic module’. This module provides a basic description of medical output using medical terms. The medical terminology module then does a simple translation of the relevant terms into a different language. This simple translation can be simply realized using a dictionary or look-up table. In one exemplary scenario, selection input can be applied to a portion of text outlining a symptom or diagnosis; an explanation module takes information from the input data and key basic module parameters to generate a detailed explanation of the symptom or diagnosis. In essence, the explanation module gives an expanded description that explains a symptom, diagnosis, treatment, medication, cure, and other relevant information. The explanation provides information more than a simple term or word. Hereinafter, the term ‘explanation module’ is interchangeable with ‘expanded module’. The expanded module then presents this expanded description in a different language. The expanded module also caters to the expertise level of the user. The explanation may also provide in so-called layman's words for the purpose of easier understanding. For example, the translation processor shows a doctor more complex information about the symptom or diagnosis, such as any scientific terminology or related data from medical studies. On the other hand, a patient is shown a simpler explanation in layperson's terms. They would be shown a common name of the symptom or diagnosis, a simple explanation, and how to treat it (e.g., take cough syrup three times a day); a visual module takes information from the medical inputs and key parameters from the basic and expanded modules to search up related pictures, videos, and other relevant forms of media. Hereinafter, the term ‘visual module’ is interchangeable with ‘advanced module’. Essentially, the advanced module gives an advanced description of the symptom or diagnosis by displaying examples of a symptom or diagnosis with visual media; a visual media search is carried out with text input from preprocessed visual input and any selection input applied to the visual media input. Keywords from the preprocessed visual and selection inputs are used as search words for the visual media search using medical data. Language translation may also apply to find videos (and even images) of a symptom or diagnosis in the preferred language. All three modules within the translation processor then generate medical outputs in their respective forms: the basic and expanded modules generate medical information as a second language output via text or audio; the advanced module generates medical output in the form of visual output.

Medical data in the present disclosure refers to sources of medical information outside of the translation processor, which is obtained from sources including the Internet (i.e., Google search), medical databases, journal articles, etc. More precisely, medical data comprises data sources (i.e., Internet search, databases, etc.) that are transferred to the translation processor via a connection with the network and application server. The translation processor uses the data sources of the medical data to form an accurate diagnosis or symptom description with multimodal language translation. All three modules of the translation processor obtain information from the medical data in order to operate. Additionally, AI may be used in tandem with medical data to automatically generate the required medical outputs. It should be noted that the multimodal translation is bi-directional. For example, the patient's input is in a first language that gets translated to output in a second language for the doctor. As the doctor speaks, the second language now becomes the input language that is translated to the patient's language, now serving as the output language. The translation processor can also translate to and from any two different content formats. The translation and description generation may occur one after another, depending on the flow of the conversation. The term ‘description generation’ refers to the generation of basic, expanded, and/or advanced descriptions as noted earlier; hereinafter, it may also be interchangeably referred to as ‘non-language translation’. With this particular aspect, there is improved communication between users (i.e., doctor and patient) and improved comprehension of a symptom, diagnosis, or other medical information.

In the second aspect of the invention, there is a method for the translation of multimedia and any applicable selection inputs to the medical terminologies and explanations in the desired language. The multimedia input or visual input can be in the form of images, video, or any other suitable visual media form. The user may select a part of an image or video that outlines a symptom; this can be done manually or automatically with software (e.g., AI) as a selection input. The translation processor, specifically its advanced module, directly obtains and processes the visual and selection inputs to perform a visual media search. The translation processor then generates the appropriate visual output, the appropriate medical terminology, and the explanation, typically in the user's desired language. It should be noted that the bi-directional translation from the first aspect also applies to visual media input being translated to the desired output language or visual media input in one language being shown as visual media output in another.

Within this aspect lies one exemplary embodiment of visual input, which involves getting a camera image of a symptom from the user's device; this is applicable if the symptom is externally visible. The user, typically the patient, points the device's camera at the body area where a symptom is present. The user can take a picture or video of said symptom. Depending on the embodiment, selection input by the user may be done manually or automatically, where the area of the image with the symptom is present gets outlined. The visual and selection inputs of the patient's symptoms are directly received and processed by the advanced module of the translation processor. The advanced module uses the captured image to make an outline of the selected area. More specifically, the advanced module uses image analysis to identify the user and the symptom using certain parameters. The advanced module then uses image segmentation to isolate the part of the image where a symptom is present, typically in the form of an outline around the area of the selection input where the symptom is present; this can be done with advanced image processing with a trained AI model. The visual media search in this exemplary embodiment is carried out based on its trained AI model; the advanced module does this using medical data for reference. The appropriate visual output, medical terminologies, and explanations are then generated for the user in the desired language. By implementing a means for translating multimedia to the appropriate outputs, there is further patient comprehension of a diagnosis, and improved communication also applies since the translation is still done with the patient's language in mind. The use of a camera image to show a symptom and carry out a visual media search also allows the patient to simply show their symptom without giving a verbal/written description.

In the third aspect of the present disclosure, AI models may be formed and trained within the software to automatically and accurately translate medical inputs to the appropriate medical outputs. Each module within the translation processor has an AI model that can be trained individually. A set of medical input data can be obtained for training the AI model, which may consist of audio, text, images, etc. The data is typically preprocessed prior to its interaction with the translation processor. At this time, each AI model determines the target parameters that will be used for generating medical output. Some exemplary parameters for the AI models to use may include, but are not limited to, the color of symptom, the texture of symptom (e.g., smoothness, folds), size and area of symptom, human expression when having symptom, etc. In some sense, the desired language can be used as a parameter for the AI model, particularly for translation from one language into another. The input data is split between a training dataset and a testing dataset. The training dataset contains most of the presented medical input data and is used to train the AI model of each module. At first, the output results (i.e., translation and visual media search) may not be accurate; however, parameters can be modified in order to fine-tune the AI model's ability to generate the appropriate output. Over time, the AI models can be trained to accurately generate the correct medical translation and associated visual media related to a symptom or diagnosis. At some point, each AI model is then tested for accuracy. Each AI model generates a translation and visual search based on the testing input dataset. Parameters are modified if the AI model gives an inaccurate translation or visual search during the testing phase. If the output during testing is accurate, then the AI model remembers the output for clinical use (diagnosis) or for continued training. It should be noted that at least one AI model can be used with the second aspect for the following: automatically capture a symptom area in a camera image; analyze the camera image or selected area; isolate the part of the picture that shows a symptom; carry out a visual media search with a segmented image of a symptom. Thanks to the use of the AI model, there is increased convenience.

In the fourth aspect of the present disclosure, the translation processor is implemented as a software application or app within a device. The translation processor's implementation acts as a system that carries out the translation method outlined in the first and second aspects; this system implementation can be interchangeably referred to as the ‘translation system’. Both a patient and a doctor can use the software to interact with each other using a device like a smartphone, laptop, desktop, etc. The software application connects to medical data, which is connected to a network and application server. The application has a graphical user interface with several pages that are similar to a typical medical appointment application. The user can log in and change language settings, country of origin, etc. Patients can select a doctor in their respective country and arrange an appointment date and time; the app notifies the doctor of this appointment. Medical history can be filled out through the app by the patient, which the doctor views and archives before the appointment. The patient can also view the customs for visiting the doctor in a particular country, such as when to arrive, cultural cues, etc. One particular set of pages within the application allows patients to outline their symptoms during or before the set appointment: one page allows patients to point out the affected body area or part using a body map; another page allows patients to pick out a symptom associated with the affected body part, and describe its intensity, frequency, and start date. This information can also be seen by the doctor using the app to form a diagnosis during or before the appointment. The doctor uses medical data to determine a diagnosis, treatment, and medication; the information can then be stored in the application to be translated and shown to the patient in their respective language. With the implementation of a software application and associated systems, there is increased convenience. To an extent, communication is also improved between doctor and patient.

In the fifth aspect of the present disclosure, a security system architecture and feature are implemented so that medical data (e.g., x-rays, test results) can be securely viewed and transferred over an application server. In relation to this aspect, the term ‘medical data’ can be interchangeably referred to as ‘medical information’ or ‘medical files’. During the transfer of medical information to and from the app and server, the data becomes encrypted, and a private key is created for the user (patient or doctor) to use in order to log into the app. The user uses a password to log into the app and server to receive their medical data. At that time, the user has to enter the private key to decrypt it and view its contents. The medical information is then downloaded to the server and database. Once it is all transferred, the user logs out to end the session; the medical data is then re-encrypted. Thanks to this security system architecture/feature, there is increased security for sensitive medical data.

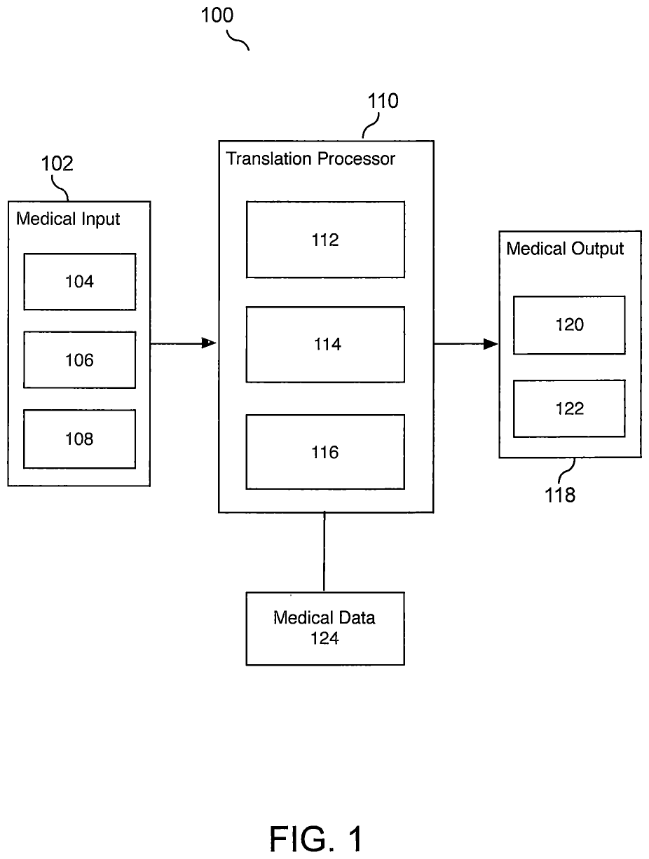

FIG. 1 illustrates a general method for multimodal translation between medical inputs and outputs. The translation method (100) involves the processing of medical inputs (102) in a first language through a translation processor (110); the medical inputs (102) comprise a first language input (104), a visual input (106), and a selection input (108). The translation processor (110) comprises three modules: a medical terminology module or basic module (112) for determining the appropriate medical terminology, giving a basic description, and giving a translation thereof; an explanation module or expanded module (114) for generating and translating an expanded description with details about a symptom or diagnosis; a visual media module or advanced module (116) that generates an advanced description by finding visual examples of a symptom or diagnosis. Medical data (124) is used to help the translation processor (110) determine a symptom or diagnosis and translate information generated by the three modules (112, 114, 116) within. Medical output (118) is then generated by the translation processor (110) in a second language; the medical outputs (118) comprise a second language output (120) and a visual output (122).

Thanks to the method (100) presented in this figure, there is improved communication between the users, namely a doctor and a patient. The modules (112, 114, 116) within the translation processor (110) provide the required information as basic, expanded, and advanced descriptions, which are typically non-language translations. The method (100) translates these descriptions from a first language to a second language as language translation. Rather than just a machine translation of what is said during an appointment, the translation processor (110) also determines the best medical term(s), a detailed explanation of said term(s), and gives visual examples (some in the translated language (e.g., video)). The modules (112, 114, 116) search for medical data (124) from external sources of information (e.g., Google search, database, journal articles, etc.) to ensure the method (100) can give an accurate description, explanation, visualization, and translation. There is also an improved understanding of a symptom, diagnosis, or other relevant medical information, mainly from the patient. During an appointment, the patient describes their symptom, which is then translated into the doctor's language by the translation processor (110). The doctor is a medical expert, so the generated descriptions can be more complex. The doctor then gives a diagnosis and explains medication or treatment. The translation processor (110) gives a more simplified translation for the patient in addition to the language translation. Because the patient and doctor can understand each other using this method (100), the flow of the conversation is smoother, and better medical care can be given.

It should be noted, however, that although the figure shows a unidirectional translation of medical inputs (102) to outputs (118), the translation between the first and second languages is bi-directional. In such a case, the second language output (120) becomes the first language input (104) as the second person speaks in a conversation. This switch between input and output language occurs as the conversation continues.

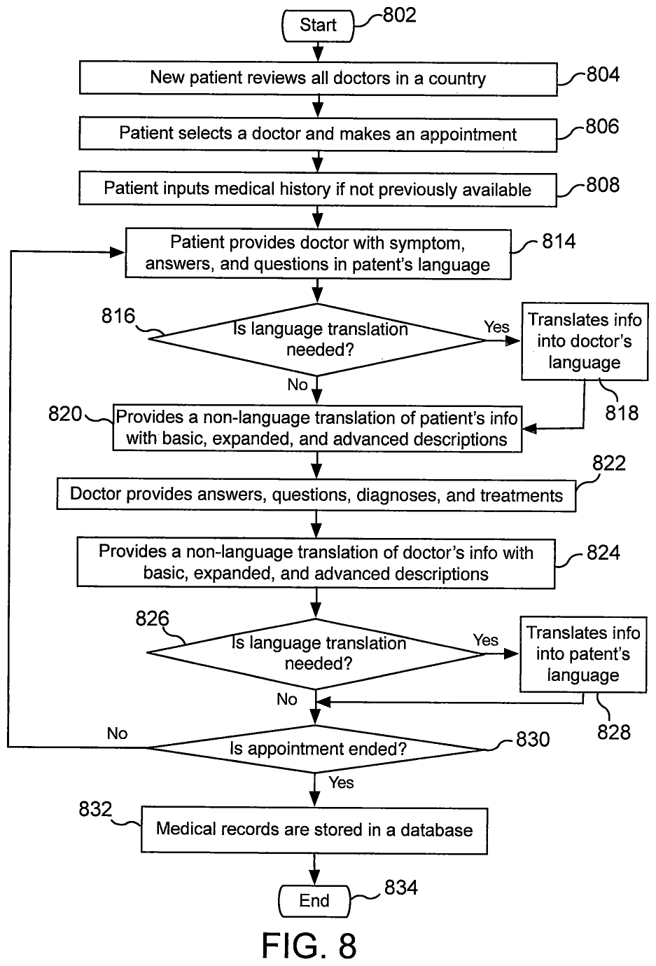

The language translation and description generation (i.e., non-language translation of basic, expanded, and advanced descriptions) during doctor-patient communication may be done simultaneously or sequentially with one after another, depending on the embodiment. In the latter case, the order of language translation and description generation depends on the flow of the conversation; this will be further demonstrated in FIG. 8.

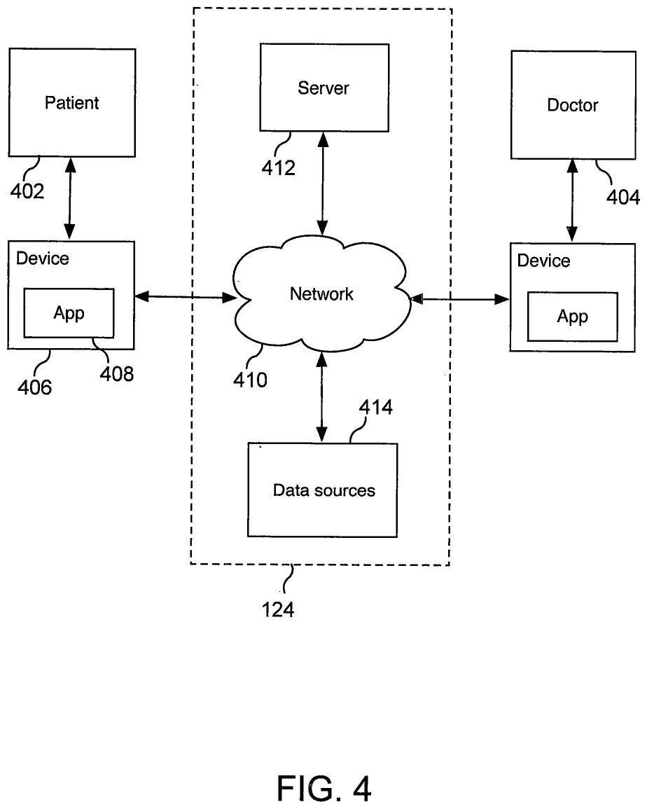

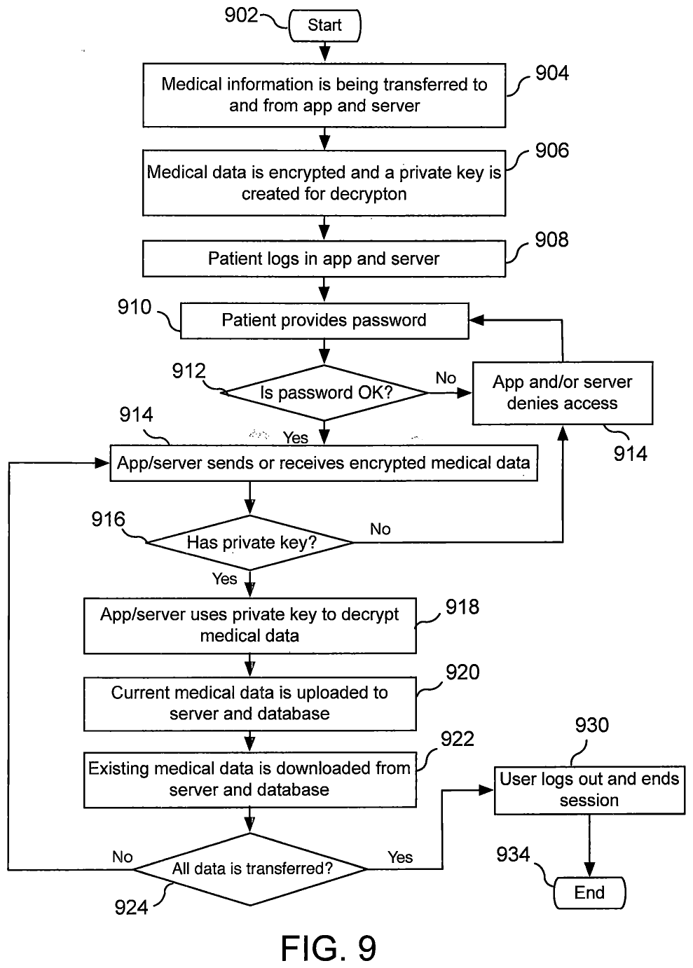

The medical data (124) needed for the description generation, and translation requires the use of a network and a server to connect to the required information Google, databases, etc.; this will be further shown in FIG. 4. The medical data (124) can also be used to transfer any medical data (e.g., conversation transcript or medical test results) that may or may not be translated, making the medical data (124) and the components of the multimodal translation method (100) interconnected with a security system architecture in the present disclosure; this will be further shown and explained in FIGS. 4 and 9.

The translation processor (110) in the preferred embodiment is integrated as a software application within a device; this will be further shown in future figures. However, the translation processor (110) can be another type of processor in another alternative embodiment. For example, the translation processor (110) may be a human interpreter in one such alternative embodiment.

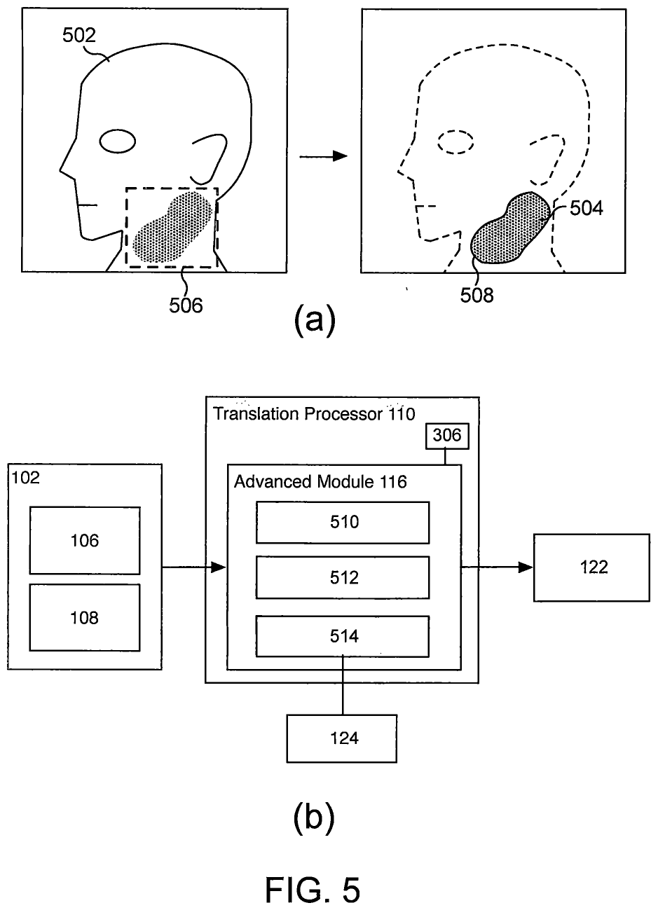

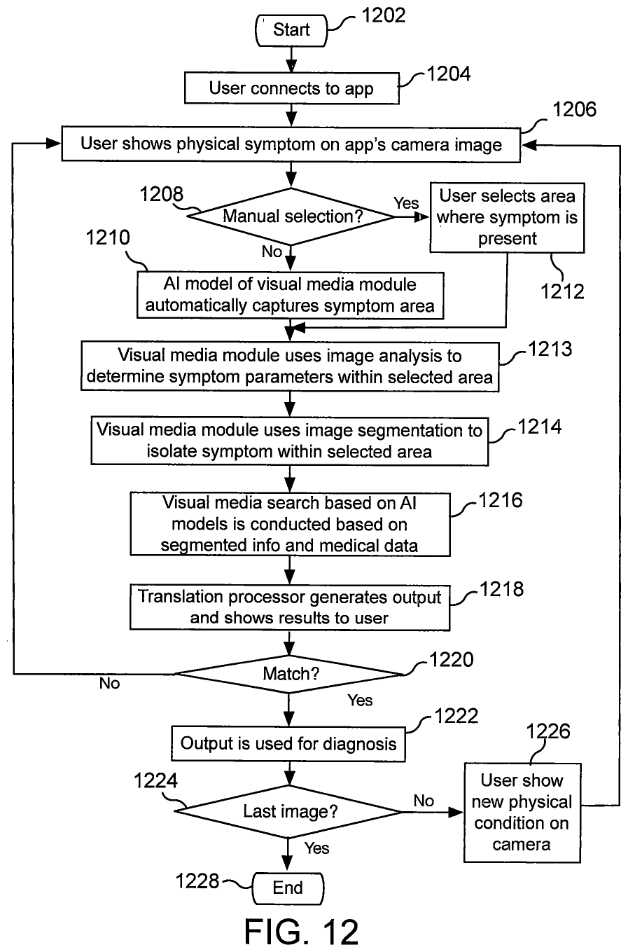

The selection input (108) is typically associated with actions done by the user within the app; this may be done manually (by the user) or automatically (by the AI), depending on the user's preference and the embodiment. In one exemplary scenario, the selection input (108) is from the user's selected language(s) and chosen format(s). In another scenario, the selection input (108) can apply to a user selecting text recorded within the appointment, highlighting it, and processing that through the translation processor (110). In one particular application of this type of input (108), the user can show a camera image of their symptom and select an area where it is present; this particular application will be further shown in FIGS. 5 and 12.

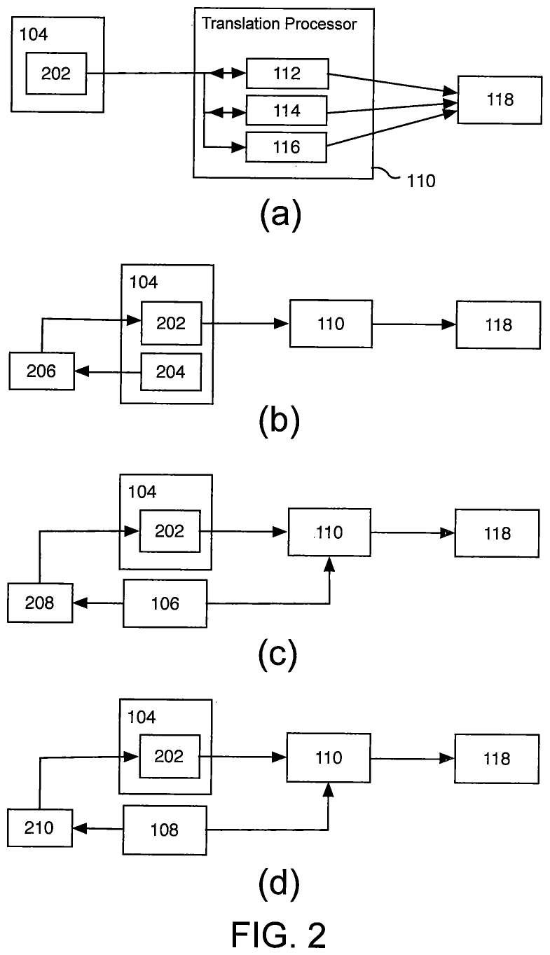

Because of the plurality of formats that can be used as medical input (102), formatting is required to convert the medical input (102) into a standardized form for language translation. Individual preprocessors convert non-text input (106, 108) into text; this will be further shown and explained in FIG. 2.

FIG. 2 illustrates how multiple modes of the inputs in a first language are translated to multiple modes of medical outputs via the multimodal translation processor. Sub-figure (a) illustrates the language translation of text (202) in a first language into multiple modes of medical outputs (118) via the multimodal translation processor (110). A first language input (104) in the form of text (202) gets processed by the translation processor (110) for language translation. The text (202) is directly received by the basic module 425 (112), which gives a basic description that is translated from the first language input (104) to a second language. The text (202) and certain basic module (112) parameters get processed in the expanded module (114) to give an expanded description. The text (202) and certain parameters from the basic module (112) and expanded module (114) get processed in the advanced module (116) to give an advanced description. All the modules (112, 114, 116) in the translation processor (110) generate medical outputs (118) in a second language.

The preferred embodiment is shown with the language translation process and preprocessing done in the particular order with the basic module (112) first, then the expanded module (114), then the advanced module (116). However, this order can be arranged differently in other alternative embodiments.

Sub-figure (b) illustrates the language translation of audio (204) sounds (e.g., voice) in a first language into multiple modes of medical outputs (118) via the multimodal translation processor (110). The audio (204) is another form of the first language input (104); it gets preprocessed by a first preprocessor (206) into text (202). All descriptions of the language translation of text (202) into medical outputs (118) from the previous sub-figure also apply here.

Sub-figure (c) illustrates the language translation of visual input (106) into multiple modes of medical outputs (118) via the multimodal translation processor (110). The visual input (106) gets preprocessed by a second preprocessor (208) into text (202). All descriptions of the language-translation of text (202) into medical outputs (118) from the previous sub-figure also apply here. Alternatively, the visual input (106) can also be directly processed by the translation processor (110) to generate the medical outputs (118).

Sub-figure (d) illustrates the language translation of selection input (108) into multiple modes of medical outputs (118) via the multimodal translation processor (110). The selection input (108) gets preprocessed by a third preprocessor (210) into text (202). All descriptions of the language-translation of text (202) into medical outputs (118) from sub-figure (a) also apply here. Alternatively, the selection input (108) can also be directly processed by the translation processor (110) to generate the medical outputs (118).

The preprocessing of non-text input (106, 108, 204) into text (202) gives the translation processor (110) a standardized format to work with. This is mainly done for translating information from one language to another, but it can also apply to description generation by the modules (112, 114, 116). ‘Text (202) is typically the simplest format to process, takes up the least amount of space, and requires fewer algorithms for the language translation process. In a sense, preprocessing into text (202) makes it more convenient to use because the translation processor (110) can handle the information more easily, making it faster to use.

In an alternative embodiment, no preprocessing is done with some or any of the non-text inputs (106, 108, 204). While the translation processor (110) can carry out the description generation; however, language translation would not be as effective, and the whole method may be slower due to the amount of data, space, and processing needed for the descriptions’ generation and the language translation thereof.

When visual input (106) and selection input (108) are preprocessed into text (202), keywords from the conversion can be used to carry out a visual media search. In some scenarios, the visual input (106) and selection input (108) do not get preprocessed and remain in their original format. One such case is when a user shows a camera image of their symptom for carrying out a visual media search for multimodal translation; this will be further shown and explained in FIGS. 5 and 12.

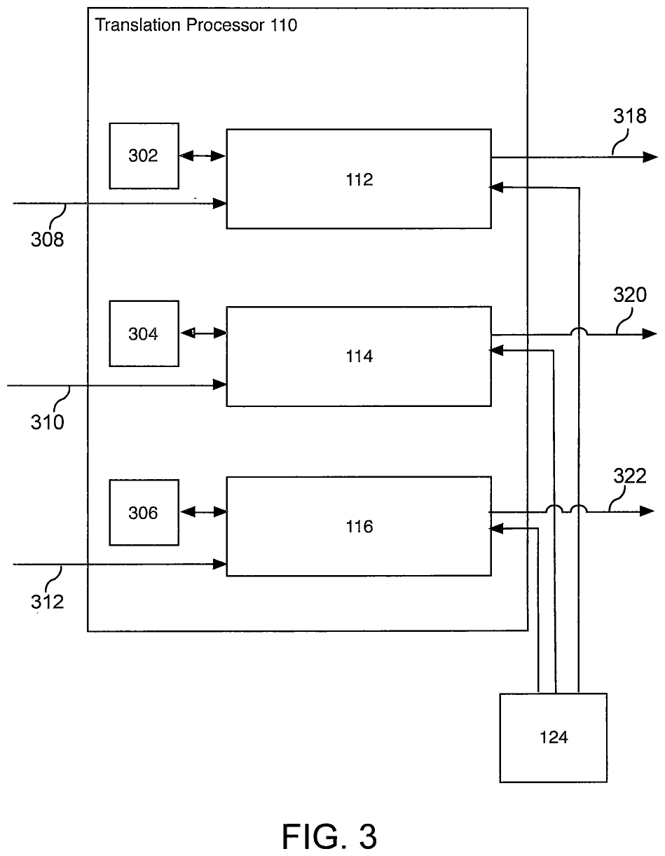

FIG. 3 illustrates an exemplary block diagram of AI processing within a multimodal translation processor. The translation processor (110) is shown with its three modules (112, 114, 116): a basic module 470 (112) receives data from a first input data path or channel (308). A first AI model (302) interacts with the basic module (112) to automatically generate the appropriate output, which travels via the first output data path or channel (318); an expanded module (114) receives data from a second input channel (310). A second AI model (304) interacts with the expanded module (114) to automatically generate the appropriate output, which travels via the second output channel (320); an advanced module (116) receives data from a third 475 input channel (312). A third AI model (306) interacts with the advanced module (116) to automatically generate the appropriate output, which travels via the third output channel (322). All the modules (112, 114, 116) receive information from the medical data (124) so that the associated AI models (302, 304, 306) can automatically generate the appropriate medical outputs.

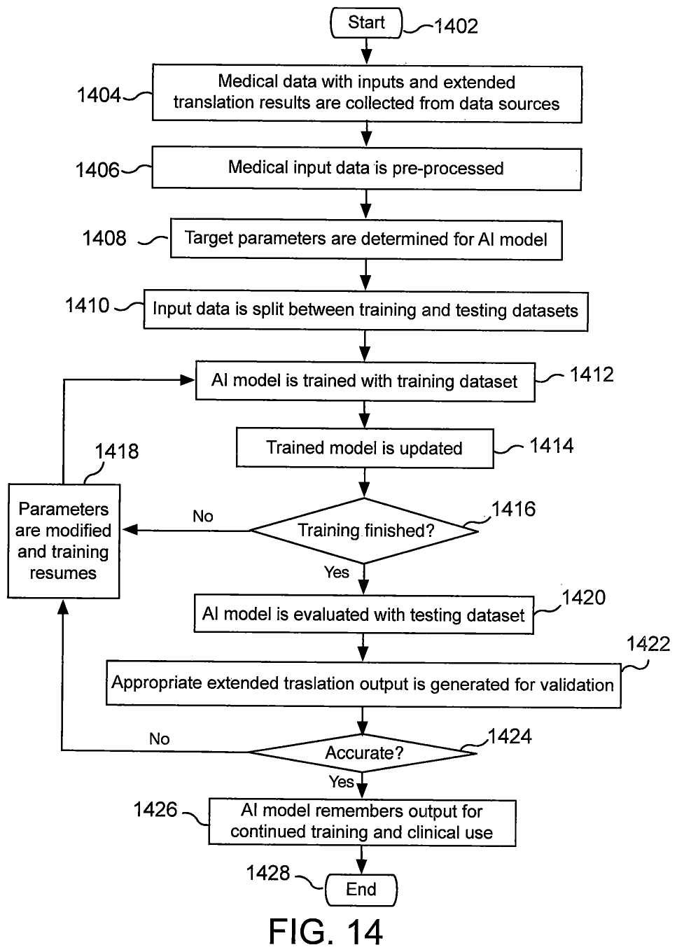

Thanks to the aspect relating to the implementation of AI models (302, 304, 306), there is increased convenience as the AI models (302, 304, 306) can automatically generate the required descriptions from each module (112, 114, 116) and translate them accordingly. It should be noted that the models (302, 304, 306) need to be trained in order to perform effectively; this process will be further shown and explained in FIG. 14.

It is presumed that preprocessing is done for the inputs prior to the description generation and language translation by the translation processor (110). However, visual input and selection input going through the advanced module (116) may not be preprocessed, and the media itself is processed by the advanced module (116) itself with the third AI model (306) to carry out a visual media search; this will be further shown and explained in FIGS. 5 and 12.

FIG. 4 illustrates an exemplary system structure design for doctor-patient communication using the multimodal translation software in the present disclosure. A patient (402) and a medical professional (404) use the multimodal translation processor as a software application or app (408) on their device (406) that connects to medical data (124). The medical data (124) comprises a network (410), a server (412), and data sources (414).

Depending on the embodiment, the device (406) used by the patient (402) and a medical professional (404) can be any electronic device (406) that can install and operate the app (408). In one preferred embodiment, the device (406) is a mobile smart device such as a smartphone. In other alternative embodiments, the device (406) may be a laptop, desktop, tablet, personal digital assistant, etc.

As noted before, the medical data (124) is the main source of reference for the app (408) to carry out its translation process. More precisely, the information itself is from the data sources (414), which include Google searches, journal articles, videos, etc. The server (412) enables information from the data sources (i.e., confidential medical data) to transfer to the users' (402, 404) device (406) for download. Such data include the patient's medical history, medication records, X-rays, test results, etc. The data that travels through the server (412) needs to be secured via encryption, which makes the system, structure design interconnected with the present disclosure's security system architecture; this will be further shown and explained in FIG. 9.

FIG. 5 illustrates an exemplary design for the media and selection inputs to be translated into visual output by the multimodal translation software. Sub-figure (a) illustrates the media and selection inputs segmenting a person's symptoms in the translation software. A user image (502) of the patient is generated on the device's camera; the user image (502) shows a rash (504) on the patient's neck. A selection (506) is made along the affected area where the rash (504) is located. The translation software then makes a symptom outline (508) of the rash (504).

It is obvious to those skilled in the art that the rash (504) in this sub-figure is an exemplary symptom; the image capture, selection (506), and symptom outline (508) are not limited to just this symptom. A myriad of other symptoms in other embodiments may be selected (506) and outlined (508) in a camera image with the translation software, provided that it is an external symptom that can be easily shown. It is also obvious to those skilled in the art that the software can capture, select (506), and outline (508) more than one symptom, typically one at a time. In an alternative embodiment, the software can simultaneously outline multiple symptoms.

Allowing the patient to select their symptom (e.g., rash (504)) from their user image (502) improves communication between the patient and doctor, especially if the two individuals do not speak the same language. Even without translation, the patient may not understand or properly explain their symptom (e.g., rash (504)). So, the software lets the patient show what is generally wrong, and the software automatically outlines (508) the affected symptom. The outlined segment (508) can be used to give more similar-looking results for the visual media search (outlined in the next sub-figure), leading to a more accurate diagnosis by the doctor, which allows for better medical care.

The selection (506) of the rash (504) or any other symptom can be done manually by the user or automatically, depending on the embodiment. In the sub-figure, the selection (506) is made by dragging a dashed rectangular box along the rash (504). The software then determines and draws the symptom outline (508) from that selection (506). In an alternative embodiment, a user may manually draw an outline (508) of their symptom image (e.g., rash (504)) on their device by dragging their finger or cursor along the symptom area.

Sub-figure (b) illustrates a method of the visual media search (514) for generating visual output (122) with the multimodal translation software. Medical inputs (102) comprising visual input (106) and selection input (108) go through the translation processor (110), specifically its advanced module (116). It should be noted that the inputs (102, 106, 108) shown refer to a captured image of a patient's symptom, as shown in the previous sub-figure. The advanced module (116) interacts with the third AI model (306) so that the visual media search (514) may be done automatically. Image analysis (510) is carried out with the visual and selection inputs (106, 108) to identify meaningful information, such as the user, a symptom, or some other key parameter. Image segmentation (512) is then carried out to isolate the selected area of a symptom within the visual and selection inputs (106, 108). A visual media search (514) is carried out with the help of medical data (124). The advanced module (116) uses the results from the visual media search (514) to generate the desired medical output (118).

As noted before, image analysis (510) determines and extracts meaningful information from the visual input (106). Oftentimes, it can be used to identify a person from their face; this seems to be done 545 with the user image (502) in the previous sub-figure. Image analysis (510) is also often used in medical imaging of internal body scans to identify organs, tissue, bone, and anomalies; such a thing can be implemented in an alternative embodiment of the present disclosure. So, image analysis (510) in the preferred embodiment determines certain parameters (e.g., color, size, anomalies on the skin, etc.) for a given symptom. The selection input (108) aids image analysis (510) by isolating the portions of the image 550 that need to be analyzed when determining what in the image is a symptom, what in the image is the patient, and everything else.

Image segmentation (512) takes information from the image analysis (510) and creates a boundary around the target objects within the image. Essentially, it labels every pixel in the said image with certain characteristics. This is commonly used in medical imaging, object detection, traffic control systems, etc. So, when the image analysis (510) determines the parameters for said symptom, the advanced module (116) then isolates the pixels of the visual input (106) with said parameters. The selection input (108) confines the area of image segmentation (512). At least one image segment is used as material for the visual media search (514) to find comparable medical data (124).

The visual output (122) as a result of the visual media search (514) allows for better communication because: (1) more accurate visual output (122) can be generated since the patient shows their symptom rather than give an explanation; (2) the doctor sees this visual output (122) from the visual media search (514) and can give a more accurate diagnosis. Additionally, the patient's understanding is also improved as the visual output (122) gives a supplementary explanation and comparison of their symptom or diagnosis. The visual output (122) strongly resembles the patient's symptoms because the visual media search (514) focuses on medical data (124) revolving around the image portion from image segmentation (512). Along with the language translation, the patient now has supplementary material to understand what they have.

It should be noted that image analysis (510), image segmentation (512), and the visual media search (514) interchangeably work together to produce the visual output (122). In one case, the image identifies key parameters relating to the user and symptom with image analysis (510). Once the symptom is identified, it gets isolated from the whole image with image segmentation (512). Image segmentation (512) is the process of partitioning a digital image into multiple image segments. The outlined image segment is then used to carry out the visual media search (514). In another scenario, the user actively segments (512) a part of the image that they believe is the symptom. The advanced module (116) then analyzes the image segment using image analysis (510) to determine what is or is not part of the symptom; the outline (508) is then made. The visual media search (514) can be done with the information from image analysis (510) and image segmentation (512). In yet another scenario, a visual media search (514) is first carried out based on the general visual input (106). The image analysis (510) can then determine the parameters that all the search results have in common. Image segmentation (512) can be used to outline the common parameters, where isolation of the related media is shown as the visual output (122).

It is obvious to those skilled in the art that the visual media search (514) can also be carried out with visual input (106) and selection input (108) preprocessed into text for language translation through the translation processor (110). As noted before, the visual media search (514) is carried out using keywords generated from the preprocessed input (106, 108). Some visual output (122) may take the form of video, so it is reasonable to presume that the visual media search (514) works in tandem with the language translation method to produce visual output (122) that is in the desired language. In an alternative embodiment of the sub-figure, preprocessed text can go through the translation processor (110) to produce visual output (122); however, when it reaches the advanced module (116), image analysis (510) and image segmentation (512) would not be involved with the preprocessed text.

While mainly done with a camera image, as shown in sub-figure (a), the direct processing of visual and selection inputs (106, 108) by the advanced module (116) can also be done with other visual media in other alternative embodiments. For example, one embodiment may involve just a random picture of someone else with red eyes (via Google Search or another source). In another embodiment, this can involve a patient's X-ray. The user or third AI model (306) selects the area with the symptom, and the advanced module (116) carries out image analysis (510), image segmentation (512), and visual media search 595 (514) based on the provided image(s).

The third AI model (306) allows the advanced module (116) to automatically carry out the visual media search (514). Because of the third AI model (306), the convenience of using the software for visual media translation is increased. Those skilled in the art know that the third AI model (306) needs to be trained prior to the visual media search (514) for clinical use; this training is further explained in FIG. 14. In an alternative embodiment, the advanced module (116) has no AI model to help with its search.

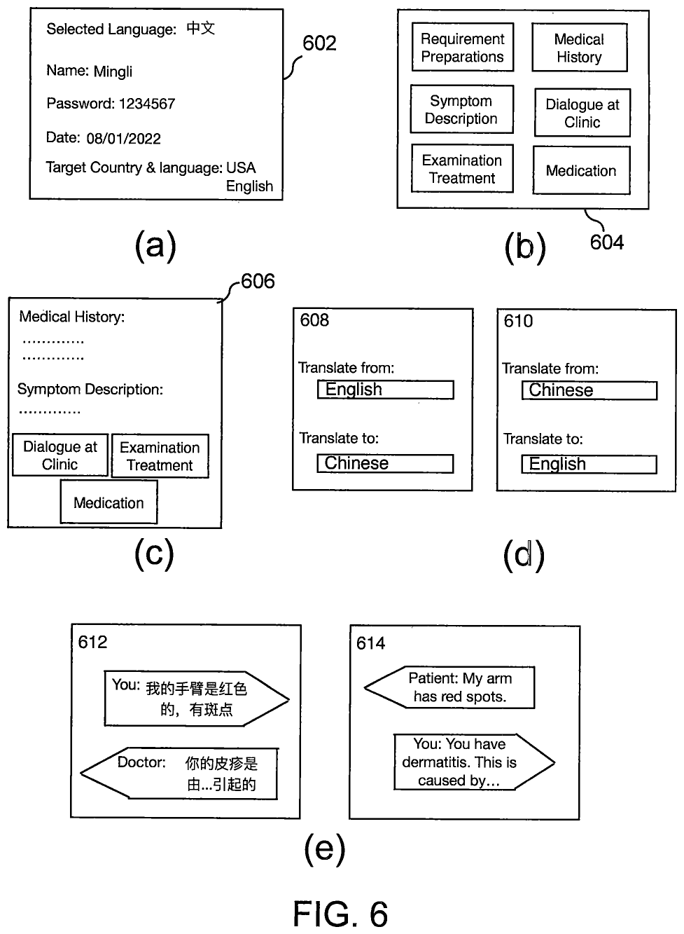

FIG. 6 illustrates the graphical user interface of the app using the translation system of the present disclosure. Sub-figure (a) illustrates the user profile/appointment page (602) from the app's graphical user interface. Here, the user selects their preferred language and type in their name and password. In the case of a patient user, they can also set an appointment date, as well as select the target country and language of their doctor visit.

Sub-figure (b) illustrates the main menu page (604). This is the default page in the app when a user logs in. The user is presented with selection icons: a ‘Requirement Preparations’ icon enables patients to view information about visiting a doctor in their selected country of appointment; a ‘Medical History’ 610 icon for typing in and viewing medical history; a ‘Symptom Description’ icon for describing symptoms; a ‘Dialogue at Clinic’ icon for enabling chat and translation during an appointment; an ‘Examination/Treatment’ icon for viewing examination results, diagnosis, and treatment; a ‘Medication’ icon for viewing the prescribed medication.

Sub-figure (c) illustrates a patient medical input page (606), where a patient can type in their medical history and their symptom descriptions (if the patient chooses to do so prior to the appointment). There are also selection icons for ‘Dialogue at Clinic’, ‘Examination/Treatment’, and ‘Medication’.

Sub-figure (d) illustrates a patient language option page (608) and a doctor language option page (610), each being shown on the respective user's device. Here, they choose the language to translate from (what the opposite party is saying) and also the language to translate to (their own language). In this sub-figure, the patient speaks Chinese, and the doctor speaks English. So, the patient language option page (608) translates dialogue from English to Chinese. The doctor language option page (610) translates from Chinese to English.

It is obvious to those skilled in the art that the options for language under the patient and doctor language option pages (608, 610) (i.e., English and Chinese) are non-limiting examples and could 625 include options for a plurality of languages.

Although both patient and doctor language option pages (608, 610) allow the users to choose each other's language to translate, an alternative embodiment allows the software application to apply the selected language from the user profile/appointment page (602) as a default language under the ‘Translate to:’ option in the language option pages (608, 610).

Sub-figure (e) illustrates an exemplary conversation between the patient and the doctor, as shown in the patient dialogue page (612) and doctor dialogue page (614). The patient gives a description in Chinese, shown through the patient dialogue page (612); this gets translated to English within the doctor dialogue page (614). The doctor responds in the doctor dialogue page (614) with a more complex diagnosis with scientific terminology. When the patient receives the doctor's words, the patient dialogue page (612) shows the translation to the patient's language and in layperson's terminology.

Thanks to the graphical user interface presented in the software application, both the patient and doctor experience increased convenience. Considerable time can be saved by doing administrative stuff on the app ahead of time rather than just prior to an appointment. For example, the medical history portion within the medical input page (606) allows the patient to fill out their medical history and potentially their symptoms days before the appointment rather than right before the patients walk in. That way, the patient has time to fill out the information properly and accurately without having to rush. The doctor can also review this information prior to the appointment, so they can better understand the patient or even make a more accurate diagnosis prior to the appointment. The translation processor would presumably be at work to translate and describe a patient's symptoms and other relevant information at this stage prior to the appointment. In any case, there can be more time for doctor-patient interaction because the administrative information is also provided beforehand.

The software application and its graphical user interface also provide improved communication for language translation and for the preparation of an appointment. Looking at the main menu page (604), the ‘Requirement Preparations’ option allows patients to view information about seeing a doctor in their chosen country, such as any needed preparation for an appointment, cultural cues, etc. This way, the patient becomes more aware of how to communicate with the doctor to get a good diagnosis and to ensure a smooth conversation without tension or misunderstandings.

It should be noted that all the content within the software's pages (602-614) and selection icons therein are non-limiting examples of application pages and selection icons for the software's graphical user interface. In other embodiments, the information shown on each page can vary and may contain different options, or there can be entirely different pages. For example, there may be a page for medication to show what medication to take after a diagnosis and an option to have it delivered to a patient's home or nearest pharmacy. In another alternative embodiment, there can be an option within the main menu page (604) where the patient can select from a list of doctors within the country of their appointment. In yet another alternative embodiment, there can also be a page for handling customer payments or scheduling different patients in time slots, making the software app usable by receptionists or administrative staff.

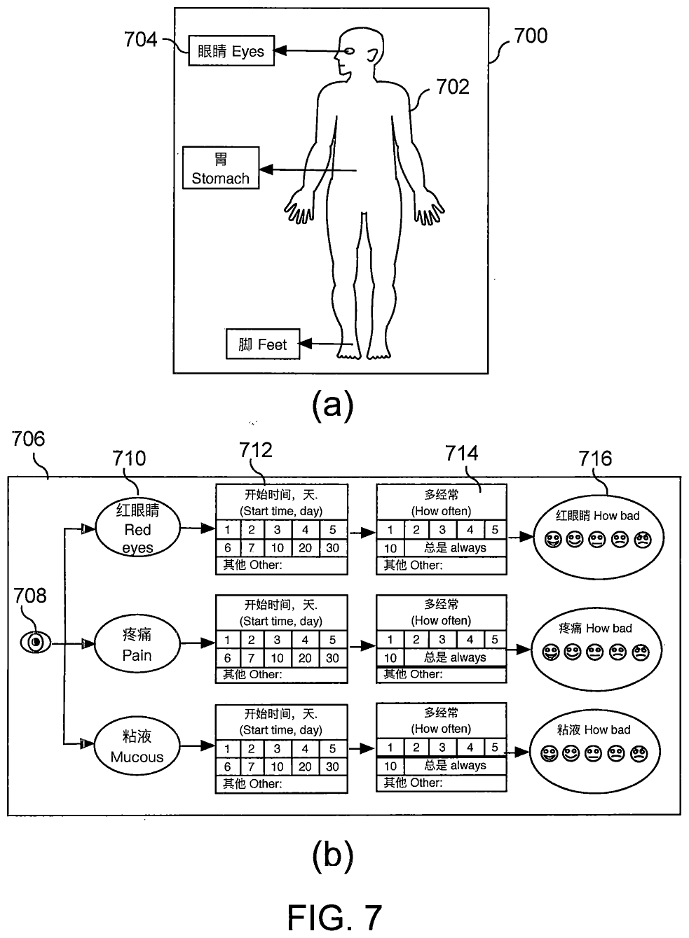

FIG. 7 illustrates the symptom description pages used for the translation system of the present disclosure. Sub-figure (a) illustrates a first symptom description page (700), which outlines a body image (702) to map out areas of the body where symptoms may appear. Each body part has body part text (704), which gives a translation of the body part in both selected languages.

The body image (702) is shown as a generic male in the sub-figure. It is obvious to those skilled in the art that the body image (702) can show a generic female within the first symptom description page (700). It is also obvious to those skilled in the art that the body image (702) can be shown with more detail (e.g., with hair) or doing some sort of action in other embodiments.

The first symptom description page (700) shows three body parts having body part text (704): the eyes, stomach, and feet. This is mainly for exemplary purposes, and it is obvious to those skilled in the art that all applicable body parts would likely have a body part text (704) for the user to select. All the present body part texts (704) are shown on the left side of the first symptom description page (700); this is exemplary, and the body part text (704) can be arranged in any way within the first symptom description page (700) in other embodiments, so long as they actually point to the correct body part on the body image (702).

Sub-figure (b) illustrates a second symptom description page (706). A chosen body part from the first symptom description page (700) is chosen by the user (i.e., the patient). In the second symptom description page (706), the chosen body part is now shown as a body part image (708) on the left side of the screen. Applicable symptoms (710) for the body part are shown on the right of the body part image (708) in the preferred and translated languages. Each symptom (710) then leads to a start date calendar (712) on the right of the symptom (710), where users can select the date the symptom (710) has started. Each symptom (710) has a frequency calendar (714) on the right of the start date calendar (712) for users to select how often it happens. Users can also rate their symptom with the intensity rating (716) on the right side of the screen, outlined with faces and associated number ratings.

The sub-figure shows all the options (708, 710, 712, 714, 716) simultaneously displayed within the second description page (706). In one embodiment, each option (708, 710, 712, 714, 716) may have a pop-up page for the patient's input. In an alternative embodiment, each option (708, 710, 712, 714, 716) is shown in separate symptom description pages.

The symptoms (710) shown for the body part image (708) in this sub-figure are an exemplary selection of symptoms (710) to choose from. It is obvious to those skilled in the art that there would be a more comprehensive list of symptoms available for each chosen body part.

The start date calendar (712) and frequency calendar (714) are shown primarily as a selection of numbers. This is for exemplary purposes, and the calendars (712, 714) can be expanded upon in other embodiments. For example, the start date calendar (712) can be shown as a proper calendar format for patients to choose from in another embodiment. The start date calendar (712) may show all the days of a particular month from Monday to Friday; the user may select a later month by clicking an arrow button beside the currently outlined month. In another embodiment, the frequency calendar (714) can be shown with options for selecting how many times a symptom happens per minute, hour, day, etc.

The intensity rating (716) is shown in the sub-figure as expressive faces and numbers. In an alternative embodiment, the intensity rating (716) can be shown as a sliding bar with one end starting with minimum intensity (e.g., no pain) to maximum intensity (e.g., maximum pain). Users would then slide a pointer along the sliding bar to express the intensity of their symptoms (710). Such a sliding bar may also be colored along a spectrum in another alternative embodiment; for example, the most painful is red on one end, and the least painful is green on the other end.

The symptom description pages (700, 706) are another set of pages within the software application's graphical user interface that increase convenience for the user, specifically the patient. The patient can use these maps to better describe their symptom without going into great detail and without needing specific medical knowledge. The patient can also describe their symptom with these pages (700, 706) prior to the appointment, allowing them to save time and get more from a doctor-patient conversation during the appointment. The doctor can view these pages (700, 706) on their end to see what a patient selects. That way, the doctor has time to analyze the information in order to make an accurate diagnosis and recommend medication/treatment at the time of the appointment.

FIG. 8 illustrates a process flowchart of the system's general use. The process starts in step (802). A new patient reviews all the doctors within a selected country in step (804). The patient selects a doctor and makes an appointment in step (806). In step (808), the patient inputs their medical history if it was not previously available. In step (814), the patient provides the doctor with their symptoms, asks questions, and answers any of the doctor's questions in the patient's language. In step (816), the process determines if language translation is needed. If it is, information from the patient's dialogue is translated into the doctor's language in step (818). In step (820), the translator processor provides a non-language translation of the patient's provided information with basic, expanded, and advanced descriptions. If no language translation is needed in step (816), then the process skips to step (820). In step (822), the doctor provides answers, asks additional questions, provides a diagnosis, and recommends treatments. In step (824), a non-language translation of the doctor's information is generated in the patient's language with basic, expanded, and advanced descriptions. In step (826), the process determines if language translation is needed. If it is, then the translation processor translates the info into the patient's language in step (828). The process then evaluates if the appointment has ended in step (830); this step (830) is also reached if language translation is not needed in step (826). If the appointment is not finished, the process goes back to step (814). If the appointment is finished, then the medical records are stored in a database (832), and the process ends (834).

FIG. 9 illustrates a flowchart for securely maintaining medical data. The process starts in step (902). In step (904), medical information or data is transferred to and from the app and server. In step (906), the medical data is encrypted, and a private key is created for decryption. The patient logs into the app and server in step (908). The patient provides a password in step (910). In step (912), the process evaluates if the password is acceptable. If it is not, the app and/or server denies access to the patient in step (914), and they must provide a new password in step (910). If the password is correct, the app/server sends or receives encrypted medical data in step (914). The process then determines if the patient has a private key in step (916). If no private key is present, then the process goes back to step (914). If the private key is present, then the app/server uses the private key to decrypt medical data in step (918). The current medical data is uploaded to the server and database in step (920). The existing medical data is downloaded from the server and database in step (922). In step (924), the process evaluates if all the data is transferred. If it is not, the process goes back to step (914). If it is, then the user logs out and ends the session in step (930). The process ends in step (934).

The flowchart in this figure outlines the security system architecture aspect, which provides increased security for sensitive medical data/information/files. This is mainly done with the encryption of all medical data going through the server and with the generation of a password and private key for access. Such data may include, but are not limited to: X-rays, lab test results, transcripts of doctor-patient a conversation during an appointment, medication prescriptions, etc. The medical information within the app gets re-encrypted and sent back to the server and database when the patient finishes viewing it and logs out. Because of the encryption via private key, access is not easily granted, and it is difficult to hack. As a result, the medical information and files are safely seen by just the target patient and the doctor.

It is obvious to those skilled in the art that once the patient (or doctor) logs out of the app and ends the session in step (930), the data gets re-encrypted and would need another private key to decrypt in another session.

The database within this process may be one stored within a cloud network or within a device's hard drive depending on the embodiment. Within a cloud network, the database can store far more data and handle more intensive workloads with fluctuating needs. On the other hand, information from a hard drive database may be accessed more securely since it is localized within a device's database.

The private key ensures that only authorized parties can decrypt the medical data. Depending on the embodiment, the same key or a different key can be used to re-encrypt the data once the user ends the session in step (930). The size of the key is non-limiting and can vary in size depending on the embodiment, although a longer key is more secure since it takes more time to be cracked (if it can be). In another alternative embodiment, the key can be a public key. In yet another embodiment, both public and private keys can be used. The public key can be used to encrypt medical information, and the private key can be used to decrypt medical information.

In yet another embodiment, only a password is used to access the medical information from the app and/or server. While it makes files easier to access, this may compromise the security of the server and security system architecture as a whole.

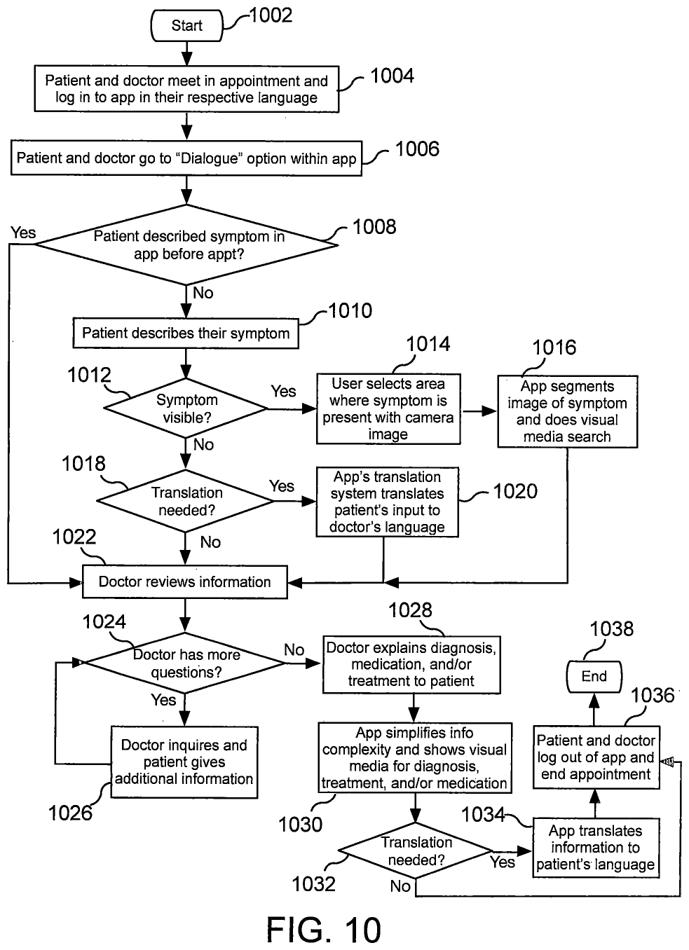

FIG. 10 illustrates a flowchart for doctor-patient communication using the application in the present disclosure. The process starts with step (1002). In step (1004), the patient and doctor meet during the medical appointment, where they will log into the app in their respective language. Both the patient and the doctor will go to the ‘Dialogue’ option within the app in step (1006). In step (1008), the process determines if the patient has described their symptoms in the app before the appointment. If the patient has, then the doctor reviews the information in step (1022). If the patient has not, then the patient 775 describes their symptom during the appointment in step (1010). In step (1012), the process determines if the symptom is visible. If the symptom is visible, then in step (1014), the patient selects the area where the symptom is present with the camera image. The app then segments the image of the symptom and does a visual media search in step (1016). The doctor then reviews the information in step (1022).

If the symptom is not visible in step (1012), then the process evaluates if a translation is needed in step (1018). If it is, then the app's translation system translates the patient's input to the doctor's language in step (1020). The doctor reviews the information in step (1022). If a translation is not needed in step (1018), then the process goes to step (1022). In step (1024), the process evaluates if the doctor has more questions. If they do, the doctor inquires, and the patient gives additional information in step (1026); the process then loops back to step (1024). If or when the doctor has no more questions in step (1024), the 785 doctor explains the diagnosis, medication, and/or treatment to the patient in step (1028). In step (1030), the app simplifies the complexity of the information and shows visual media for diagnosis, treatment, and/or medication. In step (1032), the process evaluates if language translation is needed. If a translation is needed, then the app translates the information to the patient's language in step (1034). In step (1036), the patient and doctor then log out of the app and end the appointment. If language translation is not needed in step (1032), the process skips to step (1036). The process ends in step (1038).

In the preferred embodiment of the present disclosure, the doctor and patient interact with each other in person at the doctor's clinic, as stated in step (1004). In an alternative embodiment, the patient and doctor can log in to the app and meet online via a virtual appointment.

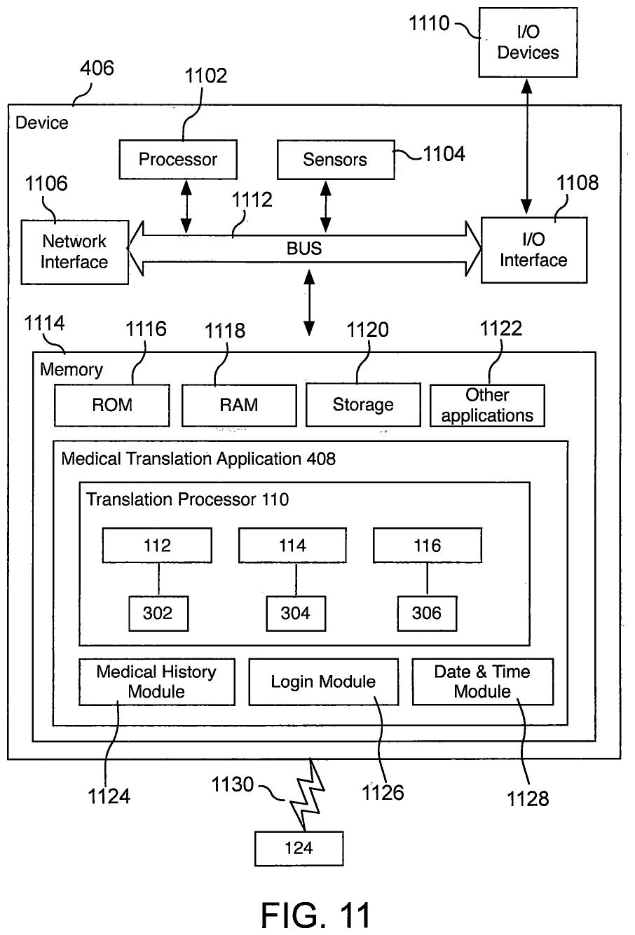

FIG. 11 illustrates a block diagram of an exemplary hardware and software implementation within a device. The device (406) is a mobile smart device in the preferred embodiment of the present disclosure. The device (406) has a processor (1102) that gives out instructions. Sensors (1104) are implemented within the device (406) to collect data from the user and their surroundings. An I/O interface (1108) allows the user to interact with the device (406) and is typically connected to I/O devices (1110) for user input and output. A network interface (1106) connects the device (406) to a network and allows it to transmit information (i.e., medical) to and from the network and application server. Using the network interface (1106), the device (406) can establish a network connection (1130) to get information from medical data (124) and connect with other devices (406) using the same app (408).

The memory (1114) includes random access memory (RAM) (1118) and read-only memory (ROM) (1116). The memory (1114) may also have hard drive storage (1120) in the form of SATA, SSD, SSHD, HDD, etc. Applications, namely the medical translation application (408) and other applications (1122), are stored and operable within the memory (114). The medical translation application (408) is operable to store the translation processor (110) with its modules: the basic module (112) with a first AI model (302); the expanded module (114) with a second AI model (304); an advanced module (116) with a third AI model (306). Additionally, the medical translation app (408) may comprise a medical history module (1124), a login module (1126), and a date and time module (1128).

The processor (1102), sensors (1104), a network interface (1106), I/O interface (1108), and memory (1114) all connect to a bus (1112) communication system. In some other embodiments, the bus (1112) system can consist of smaller buses such as: a data bus for sending information; an address bus for determining where information is sent; a control bus for determining the information's operation.

The general implementation in the figure is considered a non-limiting example for a mobile smart device. The implementation may be modified for other devices in other embodiments, such as a laptop, tablet, VR headset, personal digital assistant, etc. The implementations of the hardware components are also non-limiting, depending on the embodiment. For example, sensors (1104) within a device (406) may include, but are not limited to: a temperature sensor, a gyroscopic sensor, a GPS, a timer, etc. The network connection (1130) may include, but is not limited to: a cloud network, intranet, extranet, virtual private network (VPN), local area network (LAN), WiFi, etc. The I/O devices (1110) that generate input and output may include but are not limited to, a display, mouse, a keyboard, a monitor for display, a microphone to receive audio output, etc. The I/O devices (1110) can be separate (as shown in the figure) or integrated with the device (406), depending on the embodiment and the type of device (406).

The implementation of the software components within the medical translation application (408) is also non-limiting; this is particularly the case for the presence of the medical history module (1124), login module (1126), and date & time module (1128). In one alternative embodiment, additional modules outside those of the translation processor (110) may be present, including one for encrypting or decrypting medical information to send to the application server or database. In another alternative embodiment, the AI models (302, 304, 306) are not present within the translation processor (110).

FIG. 12 illustrates a flowchart for user input for showing a physical symptom using the system of the present disclosure. The process starts in step (1202). The user connects to the software application in step (1204). In step (1206), the user shows the physical symptom on the app's camera image. In step (1208), the process determines if this selection needs to be done manually. If not, then the visual media module's AI model automatically captures the symptom area in step (1210). If the selection in step (1208) is manual, then the process moves to step (1212), where the user instead selects the area where the symptom is present. Both steps (1210) and (1212) lead to step (1213), where the visual media module uses image analysis to determine symptom parameters within the selected area. In step (1214), the visual media module uses image segmentation to isolate the symptom within the selected area. In step (1216), a visual media search is conducted with the AI models based on the segmented information and medical data. In step (1218), the translation processor generates output and shows the results to the user. In step (1220), the process determines if the output is a match. If it is not, the process goes back to step (1206). If it is, then the output is used for the diagnosis in step (1222). In step (1224), the process determines if the last image has been presented. If not, the user shows a new physical condition on camera in step (1226), and the process goes back to step (1206). If the last image was shown in step (1224), the process ends (1228).

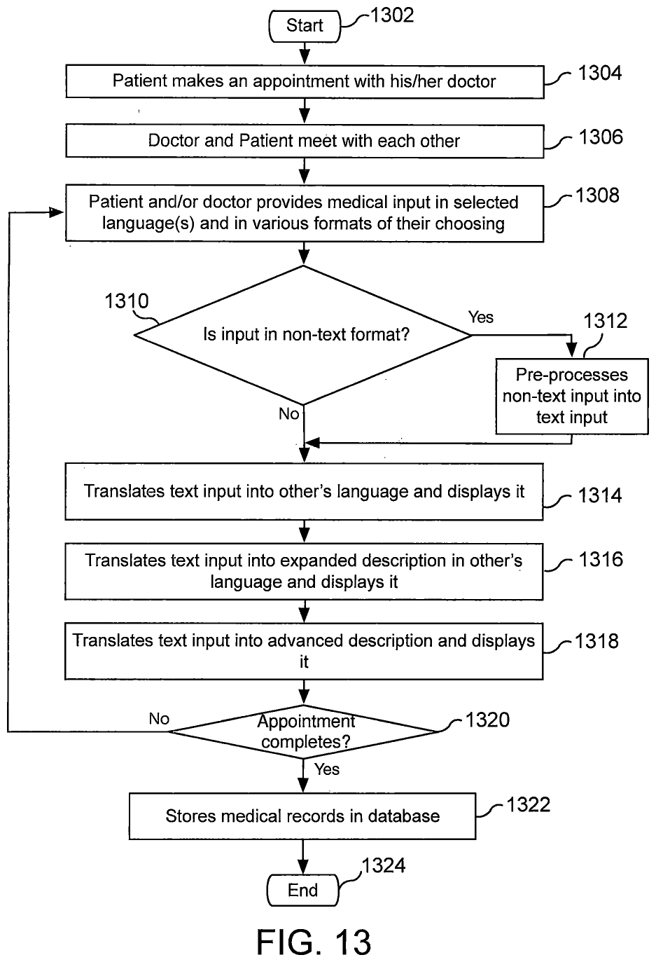

FIG. 13 illustrates a flowchart of the expanded/advanced translation process using the selection input in the present disclosure. The process starts in step (1302). The patient makes an appointment with his or her doctor in step (1304). The doctor and patient meet with each other for the appointment in step (1306). In step (1308), the patient or doctor provides medical inputs in their selected languages and in the various formats of their choosing. In step (1310), the process evaluates if the input is in non-text format. If it is not, the text input is translated into the other's language and is displayed in step (1314). If it is, then the non-text input is preprocessed into text input in step (1312) before proceeding to step (1314). In step (1316), the text input is translated into an expanded description in the other's language, which then gets displayed. In step (1318), the text input is translated into an advanced description, which then gets displayed. In step (1320), the process evaluates if the appointment is complete. If it is not, the process moves to step (1308). If it is, then the medical records from the appointment are stored in the database in step (1322). The process then ends in step (1324).

It should be noted that the language and format selections in step (808) entail exemplary forms of selection input. Other exemplary applications of selection input may include, but are not limited to: highlighting text within a software, selecting a portion of an image or video, etc.

The translation done in this figure from step (1314) to step (1318) entails both non-language translation (description generation) and language translation. It should be noted that the (non-language) translation into an advanced description in step (1318) may or may not involve language translation. In the preferred embodiment, a video that displays a symptom/diagnosis in a default desired language would be used for an advanced description. In other embodiments, a video used for the advanced description in one language may be translated to another via voice dubbing or translated subtitles in some embodiments.

As noted earlier, the order of translation of basic, expanded, and advanced descriptions in steps (1314) to (1318) is shown in a specific order. However, the order of translation may vary in alternative embodiments.

FIG. 14 illustrates a flowchart for the training and translation of the AI models within the translation processor of the present disclosure. The process starts in step (1402). Medical data with inputs and extended translation results are collected from data sources in step (1404). The medical input data is then preprocessed in step (1406). Target parameters are determined for the AI model in step (1408). Input data is split between training and testing datasets in step (1410). The AI model is trained using the training dataset in step (1412). The trained model is updated in step (1414). In step (1416), the process determines if the training is finished. If it is not, then in step (1418), the parameters are modified, and training resumes. With the modified parameters, the process goes back to step (1412). If training is considered finished in step (1416), then the AI model is evaluated with the testing dataset in step (1420). In step (1422), an appropriate extended translation output is generated for validation. In step (1424), the process determines if the extended translation output is accurate. If it is not, then the process goes to step (1418) for parameter modification and training. If the output is considered accurate in step (1424), the AI model remembers the output for continued training and clinical use (1426). The process then ends in step (1428).

The AI model training is done as a standard form of supervised machine learning. A configuration of the most advanced deep learning methods can be used here. Two common types of models are used for predicting output: a classification model is used for predicting discrete values; a regression model is used for predicting continuous values. Medical terminology is considered discrete output, so classification models are commonly used for predicting diagnoses. Naturally, the AI model in the present disclosure would be a classification model. In an alternative embodiment, a regression model can be used for predicting smaller parameters like frequency of disease or symptom, duration of disease or symptom, measurements of a symptom along a body part, etc. In another embodiment, the AI model can be in the form of unsupervised machine learning with its own process for generating output.

In step (1410), the input data used for training and testing is typically split, with 90% used as training and 10% used as testing. This split in datasets is exemplary, and the ratio of split input data may vary in other alternative embodiments. For example, the training and testing dataset can be split as 70% and 30%, respectively. However, the training dataset should be significantly larger to train the AI model better.

1. A method for translating information in a multimodal way to communicate between a first and a second user, comprising:
getting an input data that is supposed to be communicated with the second user from the first user;
wherein the input data is in text, hyperlink, pdf, audio, image, video, or a multimedia format in a first language;
providing a translation processor;
translating the input data to generate an output data for the second user by the translation processor in the following multimodal way;
wherein the output data is in text, hyperlink, pdf, audio, image, video, or a multimedia format in a second language;
wherein the output data can be in the same format as the input data but in different languages or in the different formats but in any languages;
wherein the output data is with the same, expanded or advanced meaning of the input data;
providing a data source that connects with and is accessible by the translation processor;
wherein the output data is generated based on the input data and information from the data source;
wherein the translation processor translates any single format of the input data into any single or two formats of the output data.

2. The method of claim 1, wherein at least one of the input and output data is medical information.

3. The method of claim 1, wherein the expanded meaning is an explanation of the meaning in greater detail or an easier to understand way; wherein the advanced meaning is a further related picture, video, or other multimedia format that may help understand the meaning better.

4. The method of claim 1, wherein the translation is using an AI model; wherein the AI model within the translation processor comprises: a basic module receives data from a first input data path or channel and generates data to a first output path or channel; an expanded module receives data from a second input data path or channel and generates data to a second output path or channel; an advanced module receives data from a third input data path or channel and generates data to a third output path or channel.

5. The method of claim 4, wherein the second input data path or channel can be connected to the first output path or channel; wherein the third input data path or channel can be connected to the first or second output path or channel.

6. The method of claim 4, wherein the AI model can be trained individually with a set of input data that consists of audios, texts, images, and videos; wherein the input data is split into a training dataset and a testing dataset; wherein the input data is from the data source; wherein the data source is external of the translation processor; wherein the external data source is connected through Internet; wherein the input data is medical information.

7. The method of claim 1, wherein the input data and/or data source is pre-processed and/or segmented prior to its interaction with the translation processor; wherein the interaction is AI model training or prediction.

8. The method of claim 1, wherein the input data is collected from a sensor or a user software GUI; wherein the sensor is a medical device.

9. The method of claim 1, wherein the translation is automatic and implemented in a software application; wherein the software application is an app software deployable in a smart device; wherein the smart device is a phone, laptop, desktop, tablet, or personal digital assistant.

10. The method of claim 1, wherein the data source contains medical history, medication records, X-rays, medical tests, diagnosis results, and cures.

11. The method of claim 9, wherein the first and second user knows a different language; wherein the first and second user has a difference level of knowledge and/or understanding in medical domain; wherein the multimodal translation facilitates the communication between the first and second users.

12. The method of claim 11, wherein the first and second user both use the same app software and start conversation in real-time; wherein the conversation is in the multimodal ways; wherein the multimodal translation can make a complicated medical concept easier to be understood by a patient and identify layman's description to match a medical terminology for a medical professional.

13. The method of claim 1, wherein the data source is secure stored and transmitted; wherein the data storage and transmission involve a security system architecture and feature implementation for viewing and transferring data over an application server; wherein the data is encrypted and needs a private key; wherein a user uses a password to receive their medical data and the private key to decrypt the data.

14. The method of claim 1, wherein the translation is bi-directional.

15. A software application for translating information in a multimodal way to communicate between a first and a second user, comprising:
an input data that is supposed to be communicated with the second user from the first user;
wherein the input data is in text, hyperlink, pdf, audio, image, video, or a multimedia format in a first language;
a translation processor module that translates the input data to generate an output data for the second user in the following multimodal way;
wherein the output data is in text, hyperlink, pdf, audio, image, video, or a multimedia format in a second language;
wherein the output data can be in the same format as the input data but in different languages or in the different formats but in any languages;
wherein the output data is with the same, expanded or advanced meaning of the input data;
a data source that connects with and is accessible by the translation processor;
wherein the output data is generated based on the input data and information from the data source;
wherein the translation processor translates any single format of the input data into any single or two formats of the output data

16. The software application of claim 15, wherein at least one of the input and output data is medical information; wherein the expanded meaning is an explanation of the meaning in greater detail or an easier to understand way; wherein the advanced meaning is a related picture, video, or other multimedia format that may help understand the meaning better; wherein the translation is bi-directional; wherein the input data is collected from a sensor or a user software GUI; wherein the sensor is a medical device; wherein the second input data path or channel can be connected to the first output path or channel; wherein the third input data path or channel can be connected to the first or second output path or channel; wherein the data source contains medical history, medication records, X-rays, medical tests, diagnosis results, and cures.

17. The software application of claim 15, wherein the translation is using an AI model; wherein the AI model within the translation processor comprises: a basic module receives data from a first input data path or channel and generates data to a first output path or channel; an expanded module receives data from a second input data path or channel and generates data to a second output path or channel; an advanced module receives data from a third input data path or channel and generates data to a third output path or channel; wherein the AI model can be trained individually with a set of input data that consists of audios, texts, images, and videos; wherein the input data is split into a training dataset and a testing dataset; wherein the input data is from the data source; wherein the data source is external of the translation processor; wherein the external data source is connected through Internet; wherein the input data is medical information; wherein the input data and/or data source is pre-processed and/or segmented prior to its interaction with the translation processor; wherein the interaction is AI model training or prediction.

18. The software application of claim 15, wherein the translation is automatic and implemented in a software application; wherein the software application is an app software deployable in a smart device; wherein the smart device is a phone, laptop, desktop, tablet, or personal digital assistant.

19. The software application of claim 15, wherein the first and second user knows a different language; wherein the first and second user has a difference level of knowledge and/or understanding in medical domain; wherein the multimodal translation facilitates the communication between the first and second users; wherein the first and second user both use the same app software and start conversation in real-time; wherein the conversation is in the multimodal ways; wherein the multimodal translation can make a complicated medical concept easier to be understood by a patient and identify layman's description to match a medical terminology for a medical professional.

20. The software application of claim 15, wherein the data source is secure stored and transmitted; wherein the data storage and transmission involve a security system architecture and feature implementation for viewing and transferring data over an application server; wherein the data is encrypted and needs a private key; wherein a user uses a password to receive their medical data and the private key to decrypt the data.
